(19) United States
(12) Patent Application Publication
(10) Pub. No.: US 2002/0143276 A1
(43) Pub. Date: Oct. 3, 2002

(54) Working model of the intra oral cavity

(76) Inventor: Maurice M. Ernst, Jerusalem (IL)

Correspondence Address: WELSH & FLAXMAN LLC, 2341 JEFFERSON DAVIS HIGHWAY, SUITE 112, ARLINGTON, VA, 22202, US

(21) Appl. No.: 10/105,170

(22) Filed: Mar. 26, 2002

Related U.S. Application Data
(63) Continuation-in-part of application No. 09/605,679, filed on Jun. 28, 2000, now Pat. No. 6,402,707.

(30) Foreign Application Priority Data
Jan. 15, 2002 (IL) 147654
Jun. 28, 2001 (IL) 144047

(51) Int. Cl. A61B 5/103; G06F 17/60; A61B 5/117; A61B 10/00; G06K 9/62; G06F 17/00

(52) U.S. Cl. 600/590; 128/923; 235/375; 705/4

(57) ABSTRACT

A system and a method for maintaining an association between a patient, a health provider and an insurance provider. A working model of a patient is perpetually updated to provide a basis for the insurance provider to authorize treatment plans, verify treatment quality and compliance with the guidelines of the association.

The working models of the invention can be used by forensic practitioners to perform identification of persons. Such forensic application can be applied even if only scant remains of oral tissue of a person are available for inspection.

### FIELD OF THE INVENTION

The present invention relates generally to medical care of the oral cavity of persons. The invention also relates to forensic applications involving identification of persons. More particularly the invention refers to a system for streamlining and automating the medical procedures with respect to the contractual guidelines existing between the insurance company, the patients and the health providers.

### BACKGROUND OF THE INVENTION

Commercial application of non-x-ray based imaging methods, devices, and systems in the field of dentistry, for automatically measuring, imaging, and mapping dental conditions of patients, is still significantly limited, even in view of the current rapid rate of developing and applying a wide variety of non-x-ray based imaging techniques in various other fields. The main objective, and benefit, of using automatic measuring and imaging techniques in dentistry, hereinafter also referred to as ‘dental measuring/imaging’, is to enable dental practitioners such as dentists, dental hygienists, and dental technicians to obtain highly accurate and precise realistic measurements, images, and spatial maps of intra-oral objects and features such as teeth, gum, intra-oral soft tissue, bone matter, dental undercuts, and, dental fixtures and prostheses of any kind, of dental patients, for the goal of improving procedures and processes, and decreasing costs, relating to examining, charting, diagnosing, and treating dental conditions of those patients.

Details of limitations and shortcomings associated with conventional non-imaging techniques currently used for examining, charting, diagnosing, and treating dental conditions of patients, in general, and for designing, manufacturing, fitting, and monitoring dental prostheses, in particular, are adequately described in dental literature and related prior art, for example, in U.S. Pat. No. 5,440,393, in WO 98/52493, in U.S. Pat. No. 5,273,429, in U.S. Pat. No. 4,964,770, and in U.S. Pat. No. 5,857,853.

A well known example illustrating the potentially significant utility, effectiveness, and, procedural and economic impact of successfully applying automatic measuring and imaging techniques to dentistry involves examining, charting, diagnosing, and treating dental patients requiring dental prostheses such as crowns, bridges, dentures, or implants. More specifically, data and information obtained from measuring, imaging, and mapping intra-oral objects and features can be directly used for highly accurately and cost effectively designing, manufacturing, fitting, and monitoring dental prostheses, thereby replacing currently used inaccurate, labor, material, time, and cost intensive, non-imaging techniques. Automatic dental measuring and imaging techniques are also applicable for performing various types of restorative procedures, occlusal registration, and, orthodontic and tempero mandibular joint (TMJ) dysfunction therapies.

Different categories of mechanisms, such as electrical, electronic, electro-mechanical, electro-optical, electromagnetic, radar, magnetic, magneto-mechanical, magnetic resonance, acoustic, ultrasound, sonar, photoacoustic, telemetry, and combinations of these, used for automatic three-dimensional measurement, imaging, and mapping of objects, features, and distances, are widely known and employed in various fields. The particular category of electro-optical mechanisms used in measuring and imaging techniques includes, for example, time/light in flight, laser scanning, moire, laser speckle pattern sectioning, interferometry, photogrammetry, laser tracking, and structured light or active triangulation. Specialized interferometric techniques of shearography, diffraction grating, digital wavefront reconstruction and wavelength scanning, and conoscopic holography have recently been developed as useful electro-optical measuring, imaging, and mapping techniques. Electro-optical techniques are reviewed by Chen, F., in “Overview of three-dimensional shape measurement using optical methods”, Opt. Eng. 39(1) 10-22, Jan., 2000, the contents of which are incorporated herein by reference. Several of these electro-optical techniques have been specifically applied for measuring, imaging, and mapping intra-oral objects and features. Magnetic resonance and ultrasound imaging techniques are well developed and especially applied in the medical field.

Basic in any measuring and imaging technique for accurately and precisely measuring, imaging, or mapping objects and features is the determination, sub-division, and usage of the source space and source resolution associated with the measurements and images. Measurements and images are defined in terms of global and local coordinates of the source space. Global space refers to a global coordinate space, encompassing one or more local coordinate spaces, and is at source resolution. Local space refers to a local coordinate space that is contained within global space that is also at source resolution. Accordingly, by definition, each local coordinate space and all coordinate points or positions contained therein are local with respect to the global coordinate space, whereby they can be transformed, mapped, or related to corresponding global coordinate space and global coordinate points or positions, respectively. This procedure is commonly known as registration of local coordinate space and associated local coordinate points or positions with respect to, or in terms of, global coordinate space and associated global coordinate points or positions, within source space. The registration procedure is performed by using one or more reference, fiducial, or registration, points or markers defined in the global coordinate space, which can also be associated with one or more local coordinate spaces within the global coordinate space.

Hereinafter, the terms ‘measuring system’, ‘measuring device’, ‘imaging system’, and ‘imaging device’are general, and are applicable to any category, such as those listed above, of automatic three-dimensional shape measurement, imaging, and mapping of objects and features. With respect to measuring, imaging, and mapping objects and features, the position and orientation of a measuring and imaging device such as an electro-optical measuring and imaging probe, an electromagnetic measuring and imaging probe, an ultrasound measuring and imaging probe, or a magnetic resonance measuring and imaging probe, can be characterized, described, or defined in terms of source or global coordinate space. Furthermore, for each measuring and imaging device global position, each field of view of the measuring and imaging device can be associated with a corresponding local coordinate space, within the source or global coordinate space, where the positions, orientations, and shapes or configurations, of the objects and features in each field of view of the measuring and imaging device are definable in terms of that local coordinate space. Applying a registration procedure here involves transforming or mapping local measurement and image data and information of the objects and features to global measurement and image data and information of the objects and features, using the position and orientation of the measuring and imaging device in global coordinate space as the transforming or mapping common link between global and local coordinate spaces.

Implementation of an automatic measuring and imaging technique usually includes measurement and image processing hardware and software, for automatically performing mathematical operations involved in registering local with global coordinate spaces during and/or after measurement and imaging, and for manipulating and editing measurements and images acquired in local and/or global coordinate spaces. Following these procedures, the graphical or digitized measurements and images are displayed on a display device by converting measurement and image definition from source space into device space, where device space refers to the characteristics of the device, for example, device units or pixels, by which measurements and images are displayed.

In a given automatic measuring and imaging technique, some form of one of the following three methods, object rotation method, measuring and imaging device transport method, and, fixed measuring and imaging system with multiple measuring and imaging devices method, is usually employed for the three-dimensional measuring and imaging of objects and features. In particular, in the measuring and imaging device transport method, registration of coordinate spaces, and, measurement and imaging are typically performed at a number of different fields of view of the measuring and imaging device, according to the desired extent of measuring and imaging the objects and features in the source space. Measurement and image data and information are transformed from local coordinate spaces into the global or source coordinate space using an appropriate registration procedure. Measurements and images are subsequently pieced or merged together, using an appropriate best fit algorithm, for forming composite measurements and images, or maps, of the objects and features of interest, followed by converting measurement and image, and/or map, definition from source space into device space for displaying and/or storing the measurements and images, and/or maps, of the objects and features.

Three-dimensional shape measurement, imaging, and mapping of objects and features, such as intra-oral objects and features, requires the positioning of at least one measuring and imaging device such as an electro-optical measuring and imaging probe, an electromagnetic measuring and imaging probe, an ultrasound measuring and imaging probe, or a magnetic resonance measuring and imaging probe, at different locations within source or global coordinate space, such as the oral cavity or mouth of a dental patient. Accordingly, each global location of the measuring and imaging device is associated with one or more fields of view, where each field of view is characterized or described by a corresponding local coordinate space, within the global coordinate space, and where the positions, orientations, and shapes or configurations, of objects and features of interest such as teeth, gum, intra-oral soft tissue, and dental fixtures or prostheses of any kind, are definable in terms of that local coordinate space.

Various methods are available for enabling the performing of the registration procedure between local and global coordinate spaces, where a particular method used is compatible with a particular measuring and imaging technique, such as those in the above list of categories of automatic three-dimensional shape measurement, imaging, and mapping of objects and features. Commonly used methods for performing the registration procedure are based on: (1) electro-mechanical location and orientation of the measuring and imaging device, whereby the measuring and imaging device is attached to, or are part of, a highly accurate and precise electro-mechanical system used for moving and fixing the positions of the measuring and imaging device throughout source or global coordinate space, (2) photogrammetry, also known as pattern recognition, of reference, fiducial, or registration, points or markers accurately located and fixed, and necessarily visible by the measuring and imaging device in the same field of view, or local coordinate space, of the particular objects and features being measured and imaged, (3) optical tracking of each measuring and imaging device location and/or orientation, whereby active or passive optical targets associated with local coordinate spaces defined within source or global coordinate space, are attached to, or part of, each measuring and imaging device, using an optical tracker system, and (4) hybrids of these methods.

Exemplary prior art electro-optical based methods, devices, and systems, each operating with some form of one of the above described registration procedures, for measuring, imaging and mapping intra-oral objects and features, with particular application to dentistry, are briefly summarized below. In U.S. Pat. No. 4,935,635, there is disclosed a three-dimensional measuring system particularly for dental and other space-limited uses, featuring a laser diode projecting a triangulating beam which scans the surface of intra-oral objects and features, such as a tooth, to be imaged and mapped. Preferably, in a given field of view, three teeth are scanned at a time, with resolution or accuracy of at least fifty microns. The registration procedure is based on the method of electro-mechanical location and orientation of a mostly extra-orally located imaging device including a probe, highly accurate axial stepping motors, a scanning laser beam, and photodetectors.

In U.S. Pat. No. 4,964,770, there is disclosed a process of making artificial teeth, featuring the use of interferometry, moire and laser scanning imaging methods. Three-dimensional shapes of the ground tooth and of the required artificial tooth are computed in accordance with an optic-geometric formula relating pixel parameters of intensity, background brightness, contrast, and angle. Description is provided for optically imaging only a single ground tooth and non-specific adjacent surfaces. The registration procedure is based on the method of electro-mechanical location and orientation of components of an extra-orally located imaging system including a light projector, diffraction grating, and a video camera. In U.S. Pat. No. 5,372,502 there is disclosed an optical probe and method for intra-oral three-dimensional surveying of teeth, including the use of a mobile two-dimensional LCD matrix plate pattern projection unit, optic fibers projecting patterned light beams into the oral cavity, and a CCD matrix image sensor, operating in accordance with moire, phase-shift, triangulation, and photogrammetrical techniques. Through a comparison between the undistorted pattern projected by the probe and the distorted pattern reflected from the specific area within the oral cavity, topographical information of the imaged teeth is obtained. A formal registration procedure relating local coordinate spaces of the projected and reflected patterns to source or global coordinate space of the oral cavity is not described in this disclosure, however, it is indicated that instead of using accurately controllable axial mechanical diffraction gratings, supposedly for enabling registration of local coordinate spaces, the surveying procedure is repeated a number of times, whereby accuracy of the imaging data and information is improved by iteration. Overall accuracy and precision of the method are directly dependent upon the extent of simultaneous non-movement of both the optical probe and of the patient during surveying each specific intra-oral region.

In U.S. Pat. No. 5,386,292 there is disclosed a method for correcting imaging errors due to non-optimal reflection qualities of teeth, during three-dimensional optical measurement and imaging of teeth, featuring the use of a light pattern projector and an imaging device such as a CCD matrix image sensor, operating in accordance with moire, phase-shift, and triangulation techniques. Correction is based on determining the angle of incidence of the measuring rays on the surface of a tooth. The registration procedure is based on the method of electro-mechanical location and orientation of mostly extra-orally located imaging equipment.

In U.S. Pat. No. 5,413,481 there is disclosed a method and apparatus for manufacturing fitting members, such as for a dental prosthesis, featuring the use of a CCD matrix camera for photographing moire fringes resulting from repeatedly projecting an optical groove grating in a slightly offset manner onto a prepared tooth stump. Description is provided for optically imaging only a single tooth stump. The registration procedure is based on the method of electro-mechanical location and orientation of components of an essentially extra-orally located imaging system including a projector device, a grating, mirrors, and, stepping motors and springs for fine positional control of the grating. In U.S. Pat. No. 5,440,393, there is disclosed a process and device for measuring the dimensions of a buccal cavity having upper and lower dentition, featuring the use of variable forms of a partly intra-oral optical radiation projection scanning device including light interference and deflection elements, and, an optical signal recording and digitizing system, operating in accordance with photogrammetry, triangulation, and holography techniques. Alternative registration procedures are used, including the method of electro-mechanical location and orientation of components of the scanning device, and photogrammetry. For photogrammetric registration, the position and orientation of the scanning device are determined relative to the dentition of the buccal cavity, by using an extra-orally located and fixed patient skull frame device supporting three lead balls functioning as reference, or registration, points accurately located and fixed, and necessarily visible by the scanning device, in the same field of view, or local coordinate space, of each image of the buccal cavity. In U.S. Pat. No. 5,237,998, there is disclosed a method for correlating three-dimensional images of dental arcades, for example, a tooth stump surrounded by two healthy teeth, by correlating intra-oral imaging data of a dental impression of the arcade in the occlusive position with that of the arcade without impression material, where three mutually spaced apart reference points are located and fixed in the immediate vicinity of the arcade. The registration procedure is based on the method of photogrammetry, whereby an intra-oral device featuring a clamp or bar is used for supporting three small spheres functioning as the three reference, or registration, points, accurately located and fixed, and necessarily visible by an imaging device, in the same field of view, or local coordinate space, of every image of the dental arcade. In U.S. Pat. No. 5,857,853, there is disclosed a method and system for automatically manufacturing a prosthesis to be fixed to implants in the jawbone of a patient, including an intra-oral imaging technique featuring the use of at least one camera positioned at the opened mouth of the patient for three-dimensionally imaging each implant from at least two different oral positions. The registration procedure is based on the method of photogrammetry, whereby 100-150micron diameter optical recognition points engraved into intra-oral inserts accurately located and fixed onto, and projecting above, each implant, function as the reference, or registration, points, necessarily visible by the camera, in the same field of view, or local coordinate space, of every image of an implant.

To date, the inventor is unaware of a registration procedure based on optical tracking, or based on any type of automatic tracking, featuring wired and/or wireless signal communication mechanisms, of a measuring and imaging device location and/or orientation for implementing a dental measuring and imaging technique. It is apparent in view of the prior art, that current registration procedures used for implementing dental measuring and imaging techniques are typically based on either electro-mechanical location and orientation of the measuring and imaging device in source or global coordinate space relative to the intra-oral objects and features being measured or imaged in local coordinate spaces, or, photogrammetry or pattern recognition, of reference, fiducial, or registration, points or markers accurately located and fixed in source or global coordinate space, and necessarily visible by the measuring and imaging device in the same field of view, or local coordinate space, of the particular objects and features being measured or imaged. In general, each of these registration procedures particularly used in dental measuring and imaging has significant limitations, which may be a primary reason for the current relatively worldwide low volume application of non-x-ray based commercial dental measuring and imaging systems.

Electro-mechanical based registration procedures inherently involve the design, manufacture, real time hands-on operation, and maintenance of highly accurate, precise, complex, and expensive, electro-mechanical devices, mechanisms, components, and elements for enabling proper positional and orientational control of the measuring and imaging device relative to the objects and features of the oral cavity of the dental patient. Moreover, an operator must skillfully, and timely, during a patient visit, adjust the electro-mechanics for re-positioning the measuring and imaging device in order to change fields of view, clearly required for measuring and imaging a plurality, especially a panoramic or complete set, of intra-oral objects and features located throughout the oral cavity of the patient.

A particular limitation of employing electro-mechanical based registration procedures occurs each time the patient or measuring and imaging device moves immediately prior to or during the measuring and imaging process, whereby, the operator must adjust the electro-mechanics for re-positioning the measuring and imaging device, in order to re-establish the global coordinates of the measuring and imaging device, consequently involving re-registration of local coordinates relative to the new global coordinates. Photogrammetry or pattern recognition based registration procedures do not require the extent of complex and expensive electro-mechanical hardware and/or software, however, by definition, the reference, fiducial, or registration, points or markers accurately located and fixed, and necessarily visible by the measuring and imaging device, in the same field of view, or local coordinate space, of one particular set of intra-oral objects and features being measured and imaged, must skillfully, and timely, during a patient visit, be re-positioned in the same field of view of each other particular set of intra-oral objects and features being measured and imaged by an operator. Similar to electro-mechanical based registration procedures, the re-positioning procedure is clearly required for measuring and imaging a plurality, especially a panoramic or complete set, of intra-oral objects and features located throughout the oral cavity of the patient. Another particular limitation of employing photogrammetry based registration procedures is that reference, fiducial, or registration, points or markers, need to be of proper color, dimensions and shapes or configurations in order to be properly located, fixed, and distinguishable in a variety of relatively small local coordinate spaces associated with the intra-oral objects and features. Moreover, different colors, sizes and shapes or configurations of the points or markers may need to be used for measuring and imaging the intra-oral objects and features of a single patient, according to the particular intra-oral geometric topography of the patient, and/or according to the extent of dental measuring and imaging needed by the patient.

Implementing dental measuring and imaging systems operating with electro-mechanical or photogrammetric based registration procedures requires working under the above described constrained and limiting conditions. In order to obtain and use accurate and precise panoramic intra-oral measurement and imaging data and information in basic and advanced dental applications, measurements and images from multiple fields of view need to be pieced or merged together, using an appropriate best fit algorithm, for forming composite measurements and images, or maps, of the intra-oral objects and features, usually followed by converting measurements and image, and/or map, definition from source space into device space for displaying and storing the measurements and images, and/or maps, of the intra-oral objects and features. Even for a dental measuring and imaging technique providing high resolution single field of view measurements and images of intra-oral objects and features, any error in measuring and/or calculating locations and/or orientations of the measuring and imaging device relative to the intra-oral objects and features, causes propagation error in the registration procedure, translating to propagation error in the measurement and image piecing or merging procedure, thereby decreasing accuracy, precision, and utility, of final output data and information needed by the dental practitioner.

Dental treatment covered by insurance is extremely widespread. In certain countries such as the United Kingdom all the population is automatically insured for dental treatment under the National Health Service Scheme, and most dentists work with dental insurance. The term dental treatment refers hereinafter to any procedure carried out by a certified health provider which is intended to either maintain or improve the health of the oral cavity or take precautionary steps towards such end. Such treatments relate obviously to the dentistry, orthodontics, periodontics oral medicine and oral surgery, but may relate also to other branches of the dentistry and medicinal practice which may be involved in the health of the oral cavity in general. The term treatment refers hereinafter not only to administration of drugs, surgical or any other chemical or physical intervention in the body, but also to examination, inspection, diagnosis and treatment planning, whether manual or assisted. Medical fields involved in the health of the oral cavity are typically oncology and plastic surgery. The relevant procedures include examinations, diagnosis, treatment plans, and emergency treatments done in the oral cavity or surrounding supporting tissues, both hard and soft, including the tempero-mandibular joint, the jaws and associated tissues of the lower face. The term health provider refers to any person legally licensed to do such treatments or any person recognized by the health insurer or health payer such as a licensed professional or an auxiliary person. The term insurance company refers to insurance companies, government health organizations, and any other legal body paying for dental treatment sometimes called healthcare insurers or healthcare payers. Treatment is normally paid for per capita or piecemeal per item of work, and the insurance companies or government bodies are obliged to examine and store records of patients in order to effect payment, check to control and maintain an adequate quality of treatment, and prevent fraud. At the present time this is done by written or computerised dental records that include amongst other items, a treatment plan, a record of treatment done, and other dental records such as casts, X rays and photographs and signed, and possibly stamped forms by patients and professional and auxiliary dental staff. These records are sent to the insurance companies, normally by post and copies are kept in dental offices. Occasionally, disagreements arise between the dental professional staff and the insurance companies as well as between the insured and the other parties, as to meeting the criteria set by the insurance company, or as to the need for certain treatments in the treatment plan, as well as to the quality of accomplished treatment. Current methods of examination do not enable any person other than those present in body at the examination, either prior or post treatment to see the actual state of the teeth or surrounding tissues. Current aids that can provide objective evidence upon which an assessment of a treatment can be achieved without the need for the physical presence of the recipient of the treatment, are X rays images, digital or hard copy. Such images are in effect two-dimensional distorted view that represent the hard tissues in a black and white rendition. It can not be seen if a filling is cracked or has a deficient margin, and if a filling is replaced with the same surfaces restored as previously then the X ray can not show that the filling has been replaced. Casts can not show the need or change of restorative treatment other than missing or broken teeth, and current available imaging techniques are grossly inaccurate and are unable to provide the details to enable adequate analysis of the data.

Patient identification can be achieved by a variety of known techniques. e.g by authorisation from the healthcare provider or staff on the presentation of identifying documents, or alternatively via the CPU by known identification techniques. Identification could also be achieved in a novel manner, using a dental identification method, with information from the intra oral map itself, the detail of information in the dental map being far superior to the detail available in current dental identification methods. Presently in forensic medicine tooth substance being the most hardy biological material and most resistant to degradation, dental identification may be used to confirm the identity of a corpse after other methods have been used to identify the corpse as it is a laborious and inaccurate method. Current dental identification methods match the number and type of individual teeth and position and type of restorations present to previous collected data from dental records, including but not exclusively so, dental charts, photos of the intra oral features and X rays.

Details of limitations of current techniques and methods of automatically processing acquired data for are described in U.S. Pat. No. 6,283,761, where a CPU is used to generate a diagnostic report, for diagnostic analysis. U.S. Pat. No. 5,453,009 Feldman, where a dental treatment plan is devised which has to be followed according to a sequence determined by the CPU. U.S. Pat. No. 5,930,759 describes electronic data transactions to healthcare insurers, U.S. Pat. No. 6,199,115 that describes e mail transmissions to healthcare insurers. U.S. Pat. No. 6,017,318 relates to sampling and IV drug feedback control.

Details of limitations of current techniques and methods of identifying persons from their dental records are described in U.S. Pat. No. 5,037,301 wherein a chip is inserted into a tooth to enable identification. In U.S. Pat. No. 4,031,640, a system using radiographs is used for identification. U.S. Pat. No. 5,131,844, discloses a contact digitizer, particularly for dental applications, for tracing the tooth surface.

### SUMMARY OF THE INVENTION

An aspect of the present invention is a working model of the intra oral characteristics of an insurance recipient, in which a baseline status of the oral cavity is perpetually updated. The working model is utilized in the initiation, in the conduct and in the control of medical and administrative activities associated with the medical maintenance of the oral cavity.

Another aspect of the invention is a system of maintaining the association between a patient, a health provider and an insurance provider. In accordance with the present invention the insurance provider is involved in authorization of treatment plan, treatment performance and compliance of the treatment with the guidelines of the association. Whereas the guidelines of the association are chiefly formulated by the insurance provider, the compliance with the guidelines is done automatically using images and measurements of various types, in a central or distributed computerised system.

Yet another aspect of the present invention is a forensic application, involving identification of persons. Working models of a plurality of persons is used for matching to evidence collected in the form of intra-oral tissue.

### BRIEF DESCRIPTION OF THE DRAWINGS

FIG. 1 is a flow chart describing the sequence of steps in which treatment is performed in the framework of the association between a insurance provider, a health provider and a patient;

FIG. 2 is a flow chart describing in some detail the association of the insurance provider, the patient and the health provider following a treatment session.

### DETAILED DESCRIPTION OF THE PRESENT INVENTION

### Workflow In Accordance With The Present Invention

A health provider makes use of existing three dimensional map and oral objects to compile a model of a set of intra-oral attributes. The model is utilized to initiate, conduct and report a treatment. The term treatment used hereinafter was specified above, however invention relates also to practices which are not strictly aimed at health providing or curative procedures per se. Thus, cosmetic medicine and dentistry and aesthetic dentistry are such branches of the medical practice to which the present invention apply.

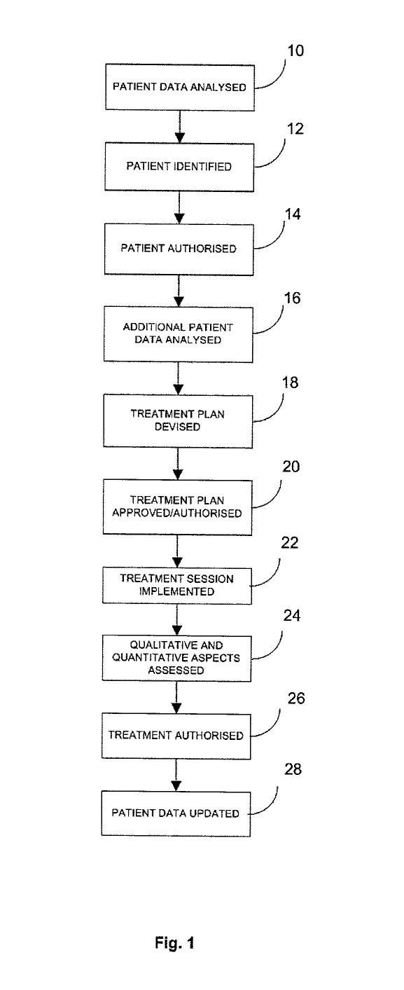

In most general terms, the process of the invention makes use of such a pre-compiled model of a set of attributes of the intra-oral objects and features of a patient. The general process is better explained by reference to FIG. 1. In step 10 patient data is analyzed such as by retrieving from a computerized database. In step 12 the patient is identified. Then, in step 14 the patient is authorized if a definite number of conditions are met. In step 16 additional patient data is analyzed. Then, in step 18 a treatment plan is devised. In step 20, the treatment plan is approved by the patient and authorized by an insurance administrative center, consequently to be implemented in step 22. In the end of the treatment session, the quality of the treatment is assessed as well as quantitative aspects in step 24. In step 26 the treatment is authorized by the insurance company, a step which may also be used as a prerequisite for transferring funds to the health provider. Last, in step 28 the patient data is updated.

### Interaction Between The Patient, The Health Provider And The Insurance Provider

A major aspect of the present invention is the formulating and the automating of the interaction among three associated parties: the patient, the health provider and the insurance provider. However, the invention may also apply to cases in which the health provider is also a health insurance provider or to such cases in which no insurance provider is involved at all.

In a typical implementation of the invention, the insurance provider is a pivotal party that formulates the rules or guidelines of the association between the patient, the health provider and the insurance provider. These rules apply to the following exemplary issues:

1. Threshold Levels for treatment initiation

A. clinical circumstances which are necessary for treatment to be covered.

B. intervals of time between renewals or repeated procedures

2. Type of insurance coverage and details of a particular policy

A. What treatment type is covered

a. Materials used in different procedures, for example silver filling or white filling of a cavity

b. Emergency treatment coverage

B. Participation/contribution to payment by the insured party

C. Treatment types and timing that do not require authorization by the insurance provider

D. Age related treatment coverage

3. Quality of treatment

Quantitative and qualitative aspects of a treatment which can be measured or assessed which are of clinical significance and the insurance provider has regulated, e.g. the fit of a prosthesis/crown, or the seal of a white filling, or the bite/occlusion of a restoration.

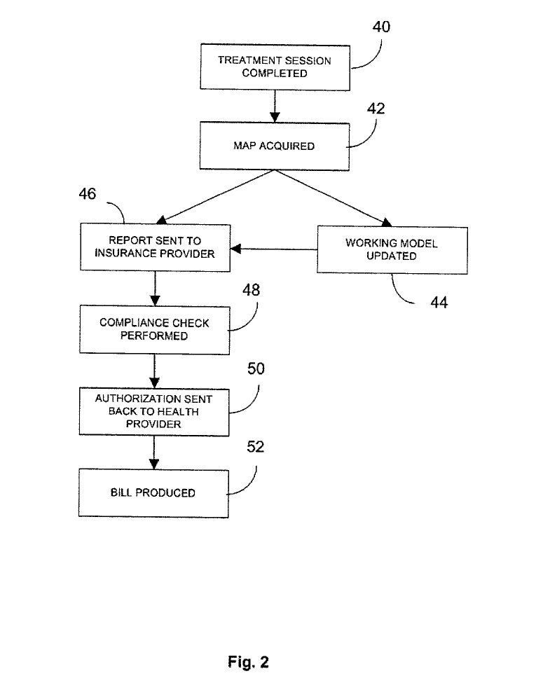

In accordance with the present invention, a treatment plan may be formulated by an automated process carried out by a computer program. The treatment plan is devised based on the updated working model and on the set of rules set by the insurance provider. The treatment plan is usually to be approved by the patient, but authorization is provided by the insurance provider consequent to the formulation of the treatment plan. Once the treatment plan is authorized, a treatment session can be initiated. When the treatment session is completed, the newly acquired images and maps of the intra oral cavity are utilized to automatically provide a quantitative and qualitative assessment of the session. This is more clearly explained with reference to FIG. 2., which describes the sequence of steps in which the interaction between the insurance provider and the other two parties are reflected. In step 40 the treatment session is completed. In step 42 various images of the intra-oral cavity are acquired, and in step 44, the working model is updated as to the status of the oral-cavity of the patient. Concurrent to the updating of the working model, a report is sent in step 46 to the insurance provider. In step 48 a treatment session compliance check is performed, based on computerized algorithm that compares the pre treatment status with respect to the post treatment status. If compliance with the guidelines (rules) of the insurance provider is met, an authorization is sent back to the health provider in step 50. In step 52 a bill is produced. The provisions of the present inventions allow the insurance provider to obtain an objective account of the treatment through which authorization can be made, rather than rely on subjective reporting or on incomplete evidence on

Thus, in the method of the present invention, the whole rapport and interaction of the healthcare insurer with the healthcare payer can be totally automated, obviating thus the need for individual checking of records, pre-authorisation and post-authorization of treatments. Consequently, the method of the invention restrains fraudulent claiming, and suppresses non payment events and lengthy correspondence between the insured patient, the professional and auxiliary dental staff and the insurance company. This leads to significant savings of time and expenses for all concerned, streamlining and making efficient the process, with eventual benefits to all parties concerned.

### The Working Model And Its Compilation And Updating

The working model of the invention includes three main components: a. Global intra oral coordinate system, b. maps and images of the intra-oral cavity registered to the global intra oral coordinate system, and c. ancillary or non spatial data.

In accordance with the present invention, the working model is the factual information source from which the procedures of the invention derive their required data. The working model of the invention resides within a computerized system, such as a desktop computer or is distributed among computers of a network. Such a computerized system apart from accommodating the working model of the invention also takes care of the communications between all the associated parties. This requires that each of the parties of the association have access to the working model whether directly or through communications means. Broad-band Internet is preferably used by remote parties of the association, in order to be able to transmit and receive images and maps of the oral cavity.

In a preferred embodiment of the invention, the baseline status of the oral cavity is composed by integrating available measurements and images of the oral cavity of the patient, typically relating to features of the teeth, gum, intra oral soft tissue, bone matter, dental undercuts, dental fixtures and prostheses. Although the baseline status can be produced at any time, it preferably reflects a starting point of a trilateral association which includes the patient, the health provider, be it a state, a clinic, and a medical insurance provider. Once the working model is compiled, it is updated as time flows. In a preferred embodiment of the invention, the baseline status is compiled before the onset of a treatment program, and the working model it is thereafter updated each time the patient is being treated or examined. The working model maintains a dynamic, updated status of the oral cavity of the patient. The ancillary data component of the working model includes non spatial information, classified into categories such as age and medical history of the patient, or physical description of entities of the oral cavities. For example, a filling maybe well defined in terms of dimensional reference to the global coordinate system, but the ancillary data of the working model defines quantitative and qualitative properties of the filling, such as creation date and composition.

### Identification Of Persons For The Prevention Of Fraud And For Forensic Applications

The dynamically updated working model of a patient, permits the use of the registered maps and images in identifying a patient or any one having a compiled working model. Whereas normally identification of a patient is carried out in the clinic by personal acquaintance or by use of an identification document, as may be dictated by the insurance provider, more subtle methods may be required at times. Thus, an insurance provider may insist on more strict identification measures if fraudulent identifications are expected.

Since the maps and images of the working model of the patient may typically contain a large amount of mapped imagery of various kinds, they can be utilized as a tool in forensic identification procedures. It is well known that the intra oral hard tissue people (and animals) is the most durable in the body as regards physical and chemical adversities. Therefore intra-oral hard tissues are the less likely to be affected. Data contained in the working model may suffice for identification of persons, even if scant remains are available for inspection. Thus, even parts of skull collected, containing only a part of the oral cavity tissues can be used by forensic practitioners to identify a person. This is carried out by matching collected evidence to registered data in a plurality of working models. The outcome of such a matching culminates in finding a best fit between a certain working model and the evidence collected. In one application approach, the matching of records, permit the accomplishment of confirmation of identification performed chiefly by other methods. Moreover, minute details available in the dental map should make possible to not only confirm identity, but to also to exploit the details as a primary identification scheme, especially when evidence as scant as a partial tooth is available. The data matching in this aspect of the invention is preferably done in an automated fashion by a computer program.

1. A working model accommodated in a computerized system, for maintaining dynamically an updated status of the oral cavity of a patient, comprising:
global intra-oral coordinate system;
registered imagery and maps of said intra oral cavity, and
ancillary data relating to said patient.

2. A working model as in claim 1, and wherein said updated status is an updated baseline status.

3. A working model as in claim 1, and wherein said computerized system is capable of communicating over a network.

4. A method for initiating the implementation of a treatment plan by an oral cavity health provider subjected to the authorization of an insurance provider, and wherein an updateable working model is used as an information source, comprising the steps of:
devising a treatment plan based on an updated working model of said patient, and
authorizing said treatment plan by a computerized program, and wherein said program implements rules of said insurance provider.

5. A method for quantitatively and qualitatively assessing the compliance of an intra oral treatment session with the rules of set by the insurance provider, comprising the steps of:
acquiring post treatment images and maps of said intra oral tissues, and
comparing said newly obtained images and maps with pretreatment images and maps, wherein said comparing is implemented by a computerized algorithm, and wherein said algorithm applies said rules to said compliance assessment.

6. A method for identifying persons wherein a working model is employed, comprising the steps of:
collecting evidence;
matching said evidence with updated working models, and
Finding a best fit.
